(19) United States
(12) Patent Application Publication
(10) Pub. No.: US 2026/0363695 A1
(43) Pub. Date: Nov. 19, 2026

(54) TRANSCATHETER SUTURE CUTTERS AND METHODS FOR USING THEM

(71) Applicant: Sutra Medical, Inc., Lake Forest, CA (US)

(72) Inventors: Thanh LE, Lake Forest, CA (US); Caitlin Martin, Foothill Ranch, CA (US); Christina Sun, Lake Forest, CA (US); Wei Sun, Foothill Ranch, CA (US)

(21) Appl. No.: 19/654,316

(22) Filed: Apr. 21, 2026

Related U.S. Application Data
(63) Continuation of application No. PCT/US24/52655, filed on Oct. 23, 2024.
(60) Provisional application No. 63/545,349, filed on Oct. 23, 2023.

(51) Int. Cl. A61B 17/04; A61B 17/00

(52) U.S. Cl. CPC A61B 17/0467; A61B 17/00234; A61B 2017/00367

(57) ABSTRACT

A suture cutter is provided that includes two nested tubular shafts, e.g., an inner shaft with a distal tip featuring a suture engagement mechanism and a cutting surface, and an outer shaft with a distal tip featuring a tubular cutting blade with a sharpened edge that is perpendicular to the cutting surface. The outer shaft is configured to slide axially along and rotate about the inner shaft. The inner and outer shafts may be flexible along at least a portion of their lengths, e.g., adjacent the distal tip. In one example, the suture engagement mechanism is a slot, large enough for the suture to fit inside, through the wall of the inner shaft from the distal tip to the cutting surface.

### RELATED APPLICATION DATA

The present application is a continuation of co-pending International Application No. PCT/US 2024/052655, filed Oct. 23, 2024, which claims benefit of U.S. provisional application Ser. No. 63/545,349, filed Oct. 23, 2023, the entire disclosures of which are expressly incorporated by reference herein.

TECHNICAL FIELD The present application generally relates to medical devices and, more particularly, to suture cutter devices and methods for using such devices, e.g., in minimally invasive medical procedures.

### BACKGROUND

Minimally invasive medical procedures are growing in popularity owing to recent advancements in clinical techniques and medical device design, and the benefits to the patients. Minimally invasive procedures offer reduced recover times, decreased post-operative pain, lower risk of complications, and smaller scars. Examples of minimally invasive procedures include laparoscopic surgery for appendectomies and gallbladder removals, arthroscopy for knee and shoulder procedures, endovascular surgery for stent placement and aneurysm repair, minimally invasive spine surgery such as microdiscectomy and endoscopic microdiscectomy, and dental procedures such as flapless implant surgery.

Minimally invasive surgical procedures are performed through small incisions which presents several challenges to surgeons. Some of the primary challenges are limited visibility, limited maneuverability, and learning to use new equipment and techniques. As such, minimally invasive medical devices and instruments require specialized designs that are small in profile (low-profile), easy to use with limited visibility, and can be operated remotely i.e., outside of the patient's body.

Traditional surgical techniques such as suturing have been adapted for minimally invasive procedures with the development of specialized tools for suturing, knot tying, and suture cutting. For example, U.S. Pat. No. 10,820,899 to George et al. discloses an arthroscopic knot pusher and suture cutter, which may be operated with a single control to both lock a suture and cut the suture tail once the knot has been pushed to the appropriate location. U.S. Patent No. 10,835,223 to Pipenhagen disclose a vascular closure device with automatic suture cutter suitable for percutaneous wound closure. U.S. Pat. No. 5,850,993 to Thompson et al. discloses a suture cutter that can cut suture tails within a patient's body with a moveable knife. U.S. Pat. No. 6,733,509 to Nobles et al. also describe a suture cutter with mechanisms to push and position a knot, and then trim the excess tails. Each of these cutter devices includes nested tubes with a slot at the distal end to capture a suture and a blade that can be advanced distally over the suture to cut it.

The cutting mechanism of these suture cutters generally requires the suture to be tensioned for the suture cutter to be effective. This makes them well suited for suture cutting applications where the path from the operator to the knot is relatively straight and short; however, they may be less effective for procedures where the access route is longer or curved where it is more difficult to create tension in the suture. Excessive tensioning of the suture could cause tearing of the surrounding tissue or dislodgement of the device depending on the application.

Therefore, devices and methods for cutting sutures in transcatheter procedures, e.g., that do not require the suture to be tensioned would be useful.

### SUMMARY

The present application is directed to medical devices and, more particularly, to suture cutter devices and methods for using such devices, e.g., in minimally invasive medical procedures.

In one example, the present application offers a unique minimally invasive suture cutter designed to easily cut sutures in transcatheter procedures. The suture cutter features a low-profile design and a torsional cutting mechanism that does not require the suture to be tensioned. Furthermore, the torsional cutting mechanism may be actuated with flexible torque-driving shafts, making the suture cutter particularly useful for navigating the vasculature.

In accordance with one example, a suture cutter is provided that includes two nested tubular shafts, e.g., an inner shaft with a distal tip featuring a suture engagement mechanism and a cutting surface, and an outer shaft with a distal tip featuring a tubular cutting blade with a sharpened edge that is perpendicular to the cutting surface. The outer shaft is configured to slide axially along and rotate about the inner shaft. The inner and outer shafts may be flexible along at least a portion of their lengths, e.g., adjacent the distal tip. In one example, the suture engagement mechanism is a slot, large enough for the suture to fit inside, through the wall of the inner shaft from the distal tip to the cutting surface.

In accordance with another example, a method is provided for cutting a suture inside a patient's body by an operator outside of the patient's body. The proximal end of the suture to be cut is inserted through the lumen of the inner shaft. The suture cutter is advanced into a patient's body to the desired cutting location. The inner shaft is manipulated outside of the patient's body to capture the suture such that a portion of the suture is in contact with the cutting surface. The outer shaft is advanced distally to sandwich the suture between the cutting blade and cutting surface. The outer shaft is rotated relative to the inner shaft to cut the suture.

In accordance with still another example, a device is provided for cutting a suture or other filament during a medical procedure that includes an elongate member comprising a proximal end, a distal end sized for introduction into a patient's body, a longitudinal axis extending between the proximal and distal ends, and a lumen extending between the proximal and distal ends sized to receive an elongate filament; a distal tip on the distal end of the elongate member comprising a distal edge and a proximal edge; an outer member slidably mounted on the distal end of the elongate member, the outer member moveable axially relative to the elongate member between a first position where a distal end of the outer member is spaced proximally from the proximal edge of the distal tip and a second position where the distal end of the outer member engages the proximal edge of the distal tip; a suture engagement mechanism on the distal tip for guiding a filament extending through the lumen from the distal edge of the distal tip to the proximal edge; a cutting element on one of the distal end of the outer member and the proximal edge of the distal tip and a cutting surface on the other of the distal end of the outer member and the proximal edge of the distal tip, the cutting element and cutting surface configured to capture the filament guided through the suture engagement mechanism when the outer member is directed from the first position to the second position; and an actuator for rotating the outer member relative to the elongate member in the second position to cut the filament between the cutting element and the cutting surface.

In accordance with yet another example, a device is provided for cutting a suture or other filament during a medical procedure that includes an elongate member comprising a proximal end, a distal end sized for introduction into a patient's body, a longitudinal axis extending between the proximal and distal ends, and a lumen extending between the proximal and distal ends sized to receive an elongate filament; a distal tip on the distal end of the elongate member comprising a distal edge and a proximal edge; an outer member slidably mounted on the distal end of the elongate member, the outer member moveable axially relative to the elongate member between a first position where a distal end of the outer member is spaced proximally from the proximal edge of the distal tip and a second position where the distal end of the outer member engages the proximal edge of the distal tip; a transverse slot extending from the distal edge of the distal tip to the proximal edge transversely relative to the longitudinal axis for guiding a filament extending through the lumen from the distal edge of the distal tip to the proximal edge; a cutting element on one of the distal end of the outer member and the proximal edge of the distal tip and a cutting surface on the other of the distal end of the outer member and the proximal edge of the distal tip, the cutting element and cutting surface configured to capture the filament guided through the suture engagement mechanism when the outer member is directed from the first position to the second position; and an actuator for directing the outer member between the first and second positions and for rotating the outer member relative to the elongate member in the second position to cut the filament between the cutting element and the cutting surface.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A and 1B show an example of a suture cutter from a front perspective view. FIG. 1A shows the suture cutter in an initial position, and FIG. 1B shows the suture cutter in a suture cutting position with a cutting blade in contact with a cutting surface.

FIGS. 2A-2C show the suture cutter of FIGS. 1A-1B in operation from a back perspective view. FIG. 2A shows a distal portion of the suture trapped in the suture engagement slot and an outer shaft of the cutter being advanced relative to an inner shaft. FIG. 2B shows the cutting blade pushing the suture against the cutting surface and the inner and outer shafts being rotated in opposite directions. FIG. 2C shows the suture in two pieces after operating the suture cutter.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Generally, the present application is directed to minimally invasive suture cutter devices that may be operated outside of a patient's body to cut a suture inside a patient's body such that a portion of the suture may be removed from the body. For example, one or more sutures may be introduced into and secured to tissue within a target location, e.g., a native valve annulus or other implantation site, to provide a rail to introduce one or more devices, e.g., a valve prosthesis or other implant, such as those disclosed in U.S. Pat. No. 11,007,057 and U.S. Publication Nos. 2021/0212824, 2021/0212825, and 2021/0298896, the entire disclosures of which are expressly incorporated by reference herein. After introducing and securing the implant, excess suture may be cut and removed, e.g., using the cutter devices and methods described herein. Alternatively, sutures may be introduced to secure a valve prosthesis or other implant to tissue at an implantation site, e.g., by advancing one or more knots over the suture, after which the excess suture material may be removed.

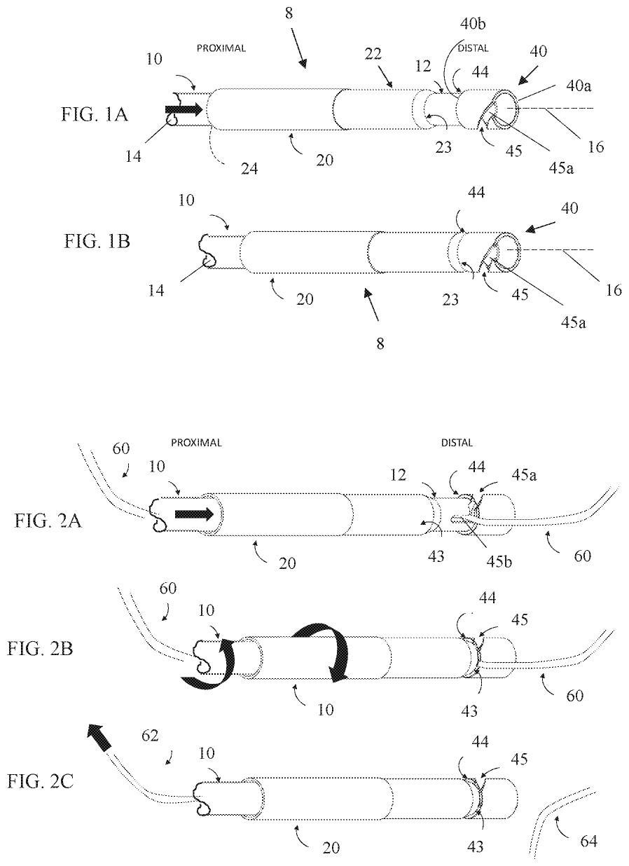

Turning to the drawings, FIGS. 1A and 1B show an example of a suture cutter 8 that includes inner and outer tubular shafts 10, 20 including proximal ends (not shown) and distal ends 12, 22 sized for introduction into a patient's body, e.g., into the patient's vasculature and/or other body lumen, to cut a suture or other elongate filament or rail, e.g., suture 60 shown in FIGS. 2A-2C. The inner and outer shafts 10, 20 may have sufficient lengths to introduce the distal ends 12, 22 into a target location within the patient's body, while the proximal ends remain outside the patient's body.

The proximal ends may be coupled to a handle or hub (not shown) that may be configured for manipulating the cutter 8 and, optionally, may include one or more actuators (also not shown) for activating the cutting element(s) on the distal ends 12, 22 to cut a suture or other filament. For example, one or sutures or other filaments may be introduced and secured to tissue at a target location, e.g., a valve annulus to guide a prosthesis or other implant into the target location. After delivering the implant, the cutter 8 may be introduced into the patient's body, e.g., over each suture, to cut the suture to allow excess suture to be removed from the patient's body, as described further elsewhere herein.

Alternatively, only one of the shafts, e.g., the inner shaft 10, may extend entirely to the proximal end, and the other shaft, e.g., the outer shaft 20, may extend proximally only partially from the distal end 22, e.g., along a distal portion of the cutter 8. In this alternative, a cable or other actuating member (not shown) may extend through a lumen 14 of the inner shaft 10, which may be coupled to an actuator on a handle or hub (not shown) to manipulate the outer shaft 20 axially and/or rotationally relative to the inner shaft 10 during operation of the cutter 8.

As shown in FIGS. 1A and 1B, the distal end 12 of the inner shaft 10 may be slidably received through a lumen 24 of the outer shaft 20, e.g., such that the distal end 12 of the inner shaft 10 extends distally beyond the distal end 22 of the outer shaft 20 along a longitudinal axis 16. The lumen 24 may accommodate the shafts 10, 20 moving axially and/or rotationally relative to one another along and/or around the axis 16. Optionally, one or more stops may be provided (not shown), e.g., within the lumen 24 and/or within a handle or hub, that may limit movement of the outer shaft 20, e.g., limiting axial movement between a proximal position, such as that shown in FIG. 1A, and a distal position, such as that shown in FIG. 1B, and/or limiting rotational movement, if desired.

In the example shown, the distal end 12 of the inner shaft 10 includes a distal tip 40 including a suture engagement mechanism 45 and a cutting surface 44, and the distal end 22 of the outer shaft 20 includes a cutting element 43 that may interact with the cutting surface 44 when the cutter 8 is actuated to severe a suture or other filament between the cutting element 43 and the cutting surface 44, as described further elsewhere herein. For example, the distal tip 40 may be a tubular member attached to and/or integrally formed around the distal end 12 of the inner shaft 10, e.g., including a distal edge 40a and a proximal edge 40b.

In the example shown in FIGS. 1A and 1B, the cutting element 43 is an annular cutting blade configured to be abutted against and/or otherwise interact with an annular cutting surface 44, e.g., to cut a suture or other filament therebetween. As shown, both the cutting blade 43 and cutting surface 44 are perpendicular to and surround the central axis 16, although alternatively, they have other orientations, if desired.

Referring to FIGS. 1A and 1B, it is contemplated that the cutting blade 43 may be configured as a tube with a sharpened distal edge extending distally from the outer shaft 20, e.g., attached to or integrally formed with the distal end 22. The cutting surface 44 may also be configured as a tube, e.g., extending proximally from the distal tip 40. In one example, the cutting blade 43 and cutting surface 44 may have about the same diameter. The cutting surface 44 is positioned distally to the cutting blade 43 such that advancing the outer shaft 20 advances the cutting blade 43 into contact with the cutting surface 44. For example, the distal end 22 of the outer shaft 20 may taper inwardly to the cutting blade 43 such that the cutting blade 43 is located immediately adjacent the outer wall of the inner shaft 10, e.g., to minimize risk of damage to tissue adjacent the cutter 8 during manipulation within the patient's body. Optionally, the cutting blade 43 and cutting surface 44 may have different diameters as long as they contact each other when directed together.

Alternatively, other configurations may be provided. For example, the cutting surface may be provided on the distal end of the outer tube and the blade may be provided on the distal tip of the inner shaft. In another alternative, both surfaces may include sharpened features that interact to cut a suture engaged between the surfaces. In addition or alternatively, the cutting blade may only extend partially around a perimeter of the distal end 22 of the outer shaft (or, alternatively, the distal tip), e.g., at least coextensive with the suture engagement mechanism such that the partial blade contacts the suture engaged between the blade and cutting surface during operation of the cutter.

As can be further seen in FIGS. 2A-2C, the suture engagement mechanism 45 may include a slot formed in the wall of the distal tip 40 and/or inner shaft 10, e.g., including a first slot portion 45a extending helically from a distal-most edge of the distal tip 40 to the cutting surface 44. The slot may have a width greater than a suture or filament to be cut, e.g., such that a suture extending through the lumen 14 of the inner shaft 10 may be received within the slot to accommodate advancing the cutter 8 over the suture, as described further elsewhere herein.

Alternatively, the first slot portion may have other configurations extending proximally from the distal edge, e.g., substantially parallel to or transverse relative to the axis 16. Optionally, the slot may include a second slot portion 45b extending circumferentially and/or axially along the wall of the inner shaft 10, e.g., proximally from the cutting surface 44 partially towards the cutting blade 43, e.g., to ensure that a suture captured in the suture engagement mechanism 45 is positioned between the blade 43 and cutting surface 44 before actuating the cutter 8.

It is contemplated that in operation, that a proximal end (not shown) of a suture 60 to be cut may be threaded through the inner lumen 14 of the inner shaft 10 and then slidably received in the slot 45. For example, after advancing the distal end 12 of the cutter 8 over the suture 60 to a target location, the proximal end of the inner shaft 10 may be manipulated to capture the suture 60 in the slot 45. For example, the inner shaft 12 (or the entire cutter 8) may be rotated to direct the suture 60 through the first slot portion 45a into the second slot portion 45b such that a portion of the suture 60 overlaps the cutting surface 44, e.g., as shown in FIG. 2A. The diagonal orientation of the first slot portion 45a may facilitate guiding the suture 60 into the second slot portion 45b by simple rotation or by rotation and advancement of the inner shaft 12. Alternatively, the suture 60 may be received in the slot 45 outside the patient's body, and then the cutter 8 may then be advanced over the suture 60, e.g., to position the distal tip 40 at a location within the patient's body where it is desired to sever the suture 60.

Once the cutter 8 is positioned at the desired location along the suture 60, the outer shaft 20 may then be advanced distally, e.g., using a first actuator on handle or hub (not shown), to sandwich the suture 60 between the cutting blade 43 and cutting surface 44, e.g., as shown in FIG. 2B. Optionally, the actuator may lock in the engaged position to secure the suture 60 between the blade 54 and cutting surface 44 without requiring further action by the operator.

The suture 60 may then be cut at the cutting surface 44 by rotating the outer shaft 20, e.g., using the first actuator or a second actuator (not shown), and either keeping the inner shaft 10 fixed or rotating the inner shaft 10 in the opposite direction, e.g. as shown in FIGS. 2B-2C. For example, the axial and rotational movement may be completed as a single sequential action by an actuator or the axial and rotational movement may be completed separately, as desired, using first and second actuators. The cut segment 62 of the suture 60 proximal to the cutting surface 44 may then be removed from the patient's body, while the segment 64 of the suture 60 distal to the cutting surface 44 remains within the patient's body, e.g., secured to tissue and/or an implant to which the suture 60 was initially introduced and/or secured.

It is contemplated that the suture engagement mechanism 45 can be configured to selectively trap and release the suture 46. Optionally, the suture engagement mechanism 45 may be configured as a slot at an approximately 45-degree angle or other angle with respect to longitudinal axis 16 of the inner shaft 10, such that the suture 60 may be trapped into the slot 45 by rotating the inner shaft 10 in a first rotative direction and may be released from the slot 45 by rotating the inner shaft 10 in a second rotative direction, e.g., if it is desired to disengage the suture 60 without cutting. For example, referring to the suture cutter 8 shown in FIGS. 1A-1B, rotating the inner shaft 10 clockwise would trap a suture 60 into the slot 45 of the suture engagement mechanism, and rotating the inner shaft 10 counterclockwise would release the suture 60. FIG. 2A shows the suture 60 trapped in the slot after rotating the inner shaft 10 clockwise.

It is contemplated that the inner and outer shafts 10, 20 may be configured as long, flexible tubes such that the suture cutter 8 can navigate a patient's vasculature, a requirement for many transcatheter procedures. For example, at least a distal portion of the inner and outer shafts 10, 20 adjacent the distal ends 12, 22 may be substantially flexible to facilitate advancing the distal ends 12, 22 through tortuous anatomy. Optionally, a proximal portion (not shown) of the inner and outer shafts 10, 20, e.g., extending distally from a handle or hub may be substantially rigid and/or semi-rigid to facilitate advancing and/or otherwise manipulating the cutter 8 from outside the patient's body.

It will be appreciated by one skilled in the arts that long, flexible tubes lack pushability, thus, it may be difficult to cut a suture 60 with conventional devices that involve pushing a cutter against a taut suture to cut it. Furthermore, it is contemplated that it may not be beneficial to the application to tension the suture, for example, if an implant is being secured with a suture locking mechanism in which the excess suture is to be removed by a transcatheter suture cutter, the device may be dislodged if the suture is tensioned excessively. The presented suture cutter operation does not require pushability or suture tension, only torqueabilty of the shafts.

In one example, at least a portion of the inner and outer shafts 10, 20 may be constructed from laser-cut hypodermic tubes or torque coils such that they are flexible and can transmit torque from the proximal end to the distal end.

It can be appreciated by one skilled in the arts that the presented suture cutter 8 does not require a cutting blade inside either the inner or outer shaft 10, 20, unlike some conventional devices. The lumen 14 of the inner shaft 10 need only be large enough to fit the suture 60. The lumen 24 of the outer shaft 20 need only be large enough to allow movement easily, e.g., axially and rotationally over the inner shaft 10. The cutting mechanism does not require the shafts 10, 20 to have much axial strength, thus the wall thickness of both shafts 10, 20 may be thin.

Optionally, the cutting surface 44 and cutting blade 43 may have the same outer diameter as the outer shaft 20. As such, the suture cutter 8 may be made with a very small profile (outer diameter), which is desirable in many transcatheter procedures.

It is fully contemplated that the features, components, and/or steps described with respect to one or more embodiments, methods, or Figures may be combined with the features, components, and/or steps described with respect to other embodiments, methods, or Figures of the present disclosure.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for cutting a suture or other filament during a medical procedure, comprising:
an elongate member comprising a proximal end, a distal end sized for introduction into a patient's body, a longitudinal axis extending between the proximal and distal ends, and a lumen extending between the proximal and distal ends sized to receive an elongate filament;
a distal tip on the distal end of the elongate member comprising a distal edge and a proximal edge;
an outer member slidably mounted on the distal end of the elongate member, the outer member moveable axially relative to the elongate member between a first position where a distal end of the outer member is spaced proximally from the proximal edge of the distal tip and a second position where the distal end of the outer member engages the proximal edge of the distal tip;
a suture engagement mechanism on the distal tip for guiding a filament extending through the lumen from the distal edge of the distal tip to the proximal edge;
a cutting element on one of the distal end of the outer member and the proximal edge of the distal tip and a cutting surface on the other of the distal end of the outer member and the proximal edge of the distal tip, the cutting element and cutting surface configured to capture the filament guided through the suture engagement mechanism when the outer member is directed from the first position to the second position; and
an actuator for rotating the outer member relative to the elongate member in the second position to cut the filament between the cutting element and the cutting surface.

2. The device of claim 1, wherein the suture engagement mechanism comprises a slot extending between the distal edge and the proximal edge of the distal tip.

3. The device of claim 2, wherein the slot comprises a first slot portion extending diagonally between the distal edge and the proximal edge.

4. The device of claim 3, wherein the slot further comprises a second slot portion extending axially along the elongate member distally from the proximal edge, the second slot portion communicating with the first slot portion such that rotation of the elongate member guides the filament through the first slot portion into the second slot portion.

5. (canceled)

6. The device of claim 1, wherein the elongate member comprises an inner tubular shaft extending between the proximal and distal ends.

7. The device of claim 6, wherein the outer member comprises an outer tubular shaft surrounding the inner shaft and extending at least partially between the proximal and distal ends.

8. (canceled)

9. The device of claim 1, further comprising a handle or hub on the proximal end of the elongate member, wherein the actuator is provided on the handle or hub.

10. (canceled)

11. The device of claim 2, wherein the actuator comprises a first actuator coupled to the outer member for directing the outer member between the first and second positions.

12. The device of claim 11, wherein the actuator comprises a second actuator for rotating the outer member relative to the elongate member, the second actuator configured for rotating the outer member relative to the elongate member only when the outer member is in the second position.

13-14. (canceled)

15. The device of claim 1, wherein the cutting element comprises a cutting blade on the distal end of the outer member.

16. The device of claim 15, wherein the cutting blade extends partially around the outer wall of the elongate member to define a first diameter, wherein the cutting surface is provided on the proximal edge of the distal tip and comprises a blunt surface defining a second diameter about the same as the first diameter, and wherein the cutting surface and the cutting blade extend around the outer surface of the elongate member substantially perpendicular to the longitudinal axis.

17-20. (canceled)

21. A suture cutter for cutting a suture, comprising:
a cutting assembly comprising an inner tubular shaft nested within an outer tubular shaft, wherein the inner shaft comprises a distal tip with a suture engagement mechanism and a cutting surface, and the outer shaft comprises a distal tip with a cutting blade that is perpendicular to the cutting surface and operable to move along the inner shaft to contact the cutting blade with the cutting surface, and a portion that can be operated by a medical professional outside of a patient's body to cut a suture inside a patient's body between the cutting blade and the cutting surface.

22. The suture cutter of claim 21, wherein the suture engagement mechanism comprises a slot that extends from a distal edge of the distal tip to the cutting surface.

23-25. (canceled)

26. The suture cutter of claim 21, wherein the suture engagement mechanism comprises a slot in the distal tip of the inner shaft that is oriented at an approximately 45-degree angle to the inner shaft.

27. The suture cutter of claim 21, wherein the suture engagement mechanism is configured to selectively trap and release the suture.

28. The suture cutter of claim 21, further comprising an actuator for rotating the outer shaft relative to the inner shaft for cutting the suture between the cutting blade and the cutting surface.

29. (canceled)

31. A method for cutting a suture inside a patient's body by an operator outside a patient's body, the method comprising:
providing a suture cutter comprising a cutting assembly comprising an inner tubular shaft nested within an outer tubular shaft, wherein the inner shaft comprises a distal tip with a suture engagement mechanism and a cutting surface, and the outer shaft comprises a distal tip with a cutting blade that is perpendicular to the cutting surface and operable to move along the inner shaft to contact the cutting blade with the cutting surface;
positioning a portion of a suture to be cut against the cutting surface;
advancing the outer shaft distally to sandwich the suture between the cutting blade and cutting surface; and
rotating the outer shaft relative to the inner shaft thereby cutting the suture.

32. The method of claim 31, wherein the suture engagement mechanism comprises a slot that extends from the distal edge of the inner shaft to the cutting surface.

33. (canceled)

34. The method of claim 31, wherein the cutting blade comprises a tube with a beveled distal edge.

35. The method of claim 31, wherein the inner and outer shafts at least adjacent the distal tip are flexible.

36-37. (canceled)
